(19) United States
(12) Patent Application Publication
(10) Pub. No.: US 2003/0003013 A1
(43) Pub. Date: Jan. 2, 2003

(54) Disinfectant composition and method for disinfecting and/or sterilising a dental unit

(75) Inventor: Franco Castellini, Bologna (IT)

Correspondence Address: Richard J. Minnich, Esq., Fay, Sharpe, Fagan, Minnich & McKee, LLP, Seventh Floor, 1100 Superior Avenue, Cleveland, OH, 44114-2518, US

(73) Assignee: CASTELLINI S.p.A.

(21) Appl. No.: 10/158,308

(22) Filed: May 30, 2002

(30) Foreign Application Priority Data
Jun. 20, 2001 (EP) 01830408.9

(51) Int. Cl. A01N 1/00; A61L 2/00

(52) U.S. Cl. 422/28; 422/34; 422/37

(57) ABSTRACT

The present invention relates to a disinfecting composition comprising a first component as peracetic ion precursor, and a second component as source of peroxide. The invention also relates to a method for disinfecting and/or sterilising the water line of a dental unit. The method comprises the steps of: preparing an aqueous solution comprising peracetic ions as active agent; introducing in the water line of the dental unit a portion of the aqueous solution comprising the peracetic ions; allowing the portion of aqueous solution comprising the peracetic ions to act; and flushing the water line with an aqueous rinsing solution.

### BACKGROUND OF THE INVENTION

The present invention relates to a disinfectant composition comprising a first component, as peracetic ion precursor, and a second component, as source of peroxide.

The invention also relates to a method for disinfecting and/or sterilizing the water line of a dental unit.

Oxidating solutions and/or compositions are used in dental surgery as disinfectants for sanitizing and disinfecting dental units and instruments such as handpieces and burs.

The biocidal compositions and/or solutions available on the market are used to disinfect external surfaces.

Prior art also teaches the use of an autoclaving method to sterilize surgical instruments and handpieces. Autoclaving is a very effective decontaminating method that will destroy bacterial colonies on instruments and/or parts of a dental unit that can be removed and placed in the autoclave.

The sterilizing action of autoclaving is applied to both external and internal surfaces.

A dental unit is equipped with a set conduits leading to handpieces and supplying each handpiece with compressed air and water and/or mixtures of air and water.

The dentist uses the handpiece to perform operations in a patient's mouth.

A bur held and revolving in a handpiece is cooled by flowing water. The bur is fitted to the handpiece and is used to drill and cut. The bur is cooled by a flow of water and air to form a spray mixture of nebulized water and compressed air.

The handpiece is thus used directly in the patient's mouth during dental treatment. As a result, bacterial contamination from the handpiece to the patient and from the patient to the handpiece is inevitable. For this reason, hygiene is a very strongly felt problem in dental surgeries. To avoid cross contamination between patients, dentists and dental hygienists must therefore adopt a series of precautions.

Bacterial contamination may occur when the dentist or hygienist switches off the spray of water and air at the end of a particular operation.

In this situation, when the flow of air and water stops, no water drips from the handpiece. This is because the minor residual pressure in the conduits tending to push the water out is counteracted by a slight pressure in the opposite direction activated to prevent the water from dripping out.

A handpiece designed to operate in this way, although it does prevent water from dripping out, may allow germs and bacteria to be sucked into the handpiece itself from the patient's mouth, causing particles of tissue from the patient's mouth to be nebulized within the handpiece. As a result, any germs and viruses present in the patient's mouth may enter the water and compressed air supply conduit through the handpiece and from there may contaminate the entire water line of the dental unit.

In an experiment to test this type of bacterial contamination, a perfectly sterilized handpiece on a dental unit was fitted with a sterile bur and supplied with sterile water from a standard container. The dental unit was used for several days, after which the water in the tank was tested for bacteriological contamination. The tests revealed traces of bacteria, indicating that micro-organisms had been transferred from the patients to the dental unit, and had contaminated all the internal conduits, the water line of the dental unit and the water tank.

This provided a clear indication that microbes and/or viruses will contaminate the handpiece and from there will spread to the conduits and container/tank.

The transfer of microbes and/or viruses from the handpiece to the entire water system is facilitated by the concentration gradients which promote the spreading of microbes within the line.

The spreading of the microbial colonies is also facilitated by the movements of the fluids within the conduits.

To overcome the problem of bacterial contamination in dental units, a variety of sanitizing protocols have been devised to introduce disinfectant in the water line.

In practice, the inside of the water conduits comes into contact with a disinfectant so that all the bacteria that may have been transferred from a patient to the conduits is completely destroyed before treatment on the next patient begins.

The removable parts of the dental unit, such as the handpieces, are sterilized in an autoclave, while the conduits forming the water line of the dental unit are disinfected according to the protocols described below.

At regular intervals, for example at the end of a day's work or during a break in work, a complete disinfecting treatment is performed on the dental unit. This essentially consists in flushing the water line with a highly sporicidal substance and then allowing the substance to stand in the conduits. The length of this type of treatment depends on the degree of decontamination required.

The substance most frequently used for this type of operation is glutaraldehyde. The sporicidal solution is pumped through the conduits and after a set time, the pump is stopped and the sporicidal solution left to stand in the conduits for a defined time. Next, the solution containing the glutaraldehyde is drained out and the water line is flushed with water.

Using glutaraldehyde as disinfectant, it is possible to disinfect a surface by leaving the glutaraldehyde in contact with the surface for approximately five minutes before flushing it out.

Alternatively, high-level disinfection can be performed by leaving the glutaraldehyde in contact with a surface for twenty to thirty minutes before flushing it out.

To reach the sporicidal level required to achieve sterility, a contact time of five hours is necessary.

This type of treatment, that is, sterilization, is therefore performed only at the end of the day. That is because performing it during normal working hours means stopping the dental unit for an excessively lengthy period of time during which no patients can be treated on that dental unit.

Indeed, the disinfection and sterilization treatment described above cannot be carried out when the dental unit is occupied by a patient.

Moreover, disinfection and sterilization cannot be carried out between one patient and the next either.

Although the disinfection and/or sterilization performed with glutaraldehyde, as described above, makes the water line of the dental unit perfectly hygienic, the water conduits are contaminated immediately as soon as work is resumed.

Moreover, the contamination is not only due to the bacteria in the patient's mouth but also to the bacteriological burden in the mains water supply which contains micro-organisms of various kinds.

The micro-organisms that inhabit the water from the mains colonize and thrive in the water line of the dental unit. The micro-organisms cling to the walls of the conduits, multiply and form a biofilm that soon becomes difficult to remove. In time, the micro-organisms contaminate the entire water line and, as they grow, the biofilm on the inside surfaces of the conduits become thicker and thicker. When the biofilm peels off the surface of the water conduit of the dental unit, the micro-organisms that have grown inside the dental unit are released into the water flow.

Tests on samples of water from the mains used for several days in a dental unit showed that the water harbored thriving bacteriological colonies of around 100,000 microbes/ml of water used.

To sum up, we can say that the water line of a dental unit is subject to the following three different types of contamination:

1. direct contamination from the patient to the dental unit;

2. contamination from the water supply to the dental unit; and

3. contamination present in the compressed air used to drive the dental instruments and for the sprays.

The technical problem underlying the present invention is the provision of a sterilizing composition and a method for disinfecting and/or sterilizing the inside surfaces of the water line of a dental unit and that overcomes the limits and disadvantages of prior art.

The applicant has solved this technical problem by formulating a special composition comprising at least a first component as peracetic ion precursor, and a second component as source of peroxide or nascent oxygen.

The present invention has for an aim to provide a sterilizing composition with a high biocidal action.

Another aim of the invention is to provide a method for rapidly and effectively disinfecting and/or sterilizing the water line of a dental unit.

### SUMMARY OF THE INVENTION

The present invention relates to a sterilizing composition as described in the relevant independent claim set out below.

The invention also relates to a method for disinfecting and/or sterilising the water lines of dental units, the method being as described in the relevant independent claim set out below.

The invention further relates to the use of a solution comprising peracetic ions as active agent for disinfecting and/or sterilising dental units, and having the characteristics described in the relevant independent claim set out below.

Other preferred embodiments of the invention are described in the dependent claims below.

Other technical characteristics of the invention appear in the detailed description below, which highlights the advantages of the invention.

### DESCRIPTION OF THE PREFERRED EMBODIMENTS

The composition that forms the subject-matter of the present invention is a solid-state composition, preferably a powder.

Advantageously, the composition may be in the solid state and may take the form of a lozenge or tablet.

Preferably, a first portion of the tablet contains the aforementioned first component and additives, as required, while a second portion of the tablet contains the aforementioned second component and additives, as required.

The composition forming the subject-matter of the present invention comprises a first component as peracetic ion precursor or peracetic acid, selected from the compounds whose general formula (I) is the following:

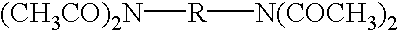

where R is a C₂-C₆ alkyl; and a second component as peroxide source or nascent oxygen, that may be a perborate or percarbonate of an alkali metal or alkaline earth metal.

Preferably, the second component is a perborate of sodium, a percarbonate of sodium or O₂ active oxygen.

Advantageously, R is ethyl and the first component is called TAED (tetra acetyl ethylene diamine); R is propyl and the first component is called TAPD (tetra acetyl propylene diamine).

In addition to the aforementioned components, the composition may include additives such as stabilizers, ion sequestrants and coformulants normally used in the preparation of tablets or lozenges such as, for example, a system to stabilize the pH values of the solid composition and to prevent the first and second components from decomposing.

As pH stabilizers, acid substances such as phosphonic acid or malonic acid may be used.

The composition forming the subject-matter of the present invention is soluble in water, preferably in water warmed to a temperature of between 30 and 40□C with a pH between 7 and 8, even if it is subsequently used at ambient temperature.

When dissolved in an aqueous solution, the composition gives rise to a reaction which, in a period of time ranging from 5 to 15 minutes, stabilizes to the following equilibrium:

NaBO₃+H₂O⇄H₂O₂+sodium borate 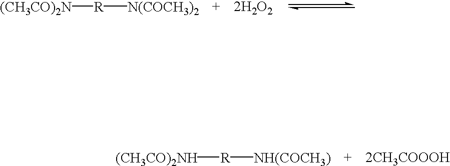
CH₃COOOH⇄CH₃COOO⁺+H⁺

where R is a C₂-C₆ alkyl group.

Alternatively, the hydrogen peroxide source may be obtained from sodium percarbonate such as, for example:

Na₂CO₃.nH₂O₂+H₂O---->nH₂O₂+sodium carbonate.

The aqueous solution comprising the first and second components has disinfecting and sterilising properties. In particular, the composition comprising peracetic ions or peracetic acid has been found to have a fungicidal, bacteriocidal, sporicidal and virocidal action.

The solution comprising peracetic acid may be stabilized by adding a suitable stabilizing agent capable of preventing the chemical degradation of hydrogen peroxide or peracetic ions. In addition, the solution may comprise a suitable sequestrant such as EDTA and a suitable surfactant.

The solution comprising peracetic ions in equilibrium with the peracetic acid produced in the solution can be advantageously used in a method for disinfecting and/or sterilising a dental unit, the solution being applied at ambient temperature in particular to disinfect and/or sterilize the inside surfaces of the water line of the dental unit.

The method forming the subject-matter of the invention comprises the steps of:

preparing an aqueous solution comprising peracetic ions as active agent;

introducing in the water line of the dental unit a portion of the aqueous solution comprising peracetic ions;

allowing the portion of aqueous solution introduced in the water line to act; and

flushing the water line with an aqueous rinsing solution.

Preferably, the step of preparing the aqueous solution comprising peracetic ions in the presence of peracetic acid as active agent further comprises the following steps:

placing in a suitable container a defined quantity of water preferably at a temperature of between 30 and 40□C and having a steady pH value of between 7 and 8;

adding to the water a quantity of the sterilising composition according to the present invention in the form of powder or, alternatively, in the form of a pre-dosed tablet to obtain a defined concentration of peracetic ions in solution;

agitating to obtain a solution and connecting the container to the water line of the dental unit.

Preferably, the step of introducing the solution is performed with the same solution at ambient temperature.

According to the method, the step of introducing the solution containing peracetic ions is followed by a pause to let the peracetic ions act within the water line of the dental unit.

Compared to treatments effected with glutaraldehyde, the results are surprising. Indeed, a high level disinfection of the water line of the dental unit can be performed in just five minutes and high efficiency sterilization in just ten minutes.

That means the method according to this invention can be conveniently used to disinfect and/or sterilize between one patient and the next during the course of a day's work, providing an effective and safe sanitizing protocol applicable not only between patients but also at the end of the day.

As a result, all patients treated during the course of the day are effectively safeguarded against cross infection.

Preparing a sufficient quantity of solution containing peracetic ions ensures that the disinfecting and/or sterilizing treatment can be performed at any time during the course of the 24 hours in a day or even over a period of more than one day since the solution, if suitably stabilized, is effective for two or more working days.

What is claimed

1. A solid composition comprising:
a first component that may be any compound whose general formula (I) is the following: 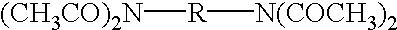
where R is a C₂-C₆alkyl group;
a second component that may be a perborate or percarbonate of alkali metals or alkaline earth metals, or O₂ active oxygen; and
one or more additives and coformulants.

2. The composition according to claim 1, wherein the first component is a compound of formula (I) where R is ethyl C₂ or R is propyl C₃; and wherein the second component is a perborate of sodium or a percarbonate of sodium.

3. The composition according to claim 2, in the form of a tablet or lozenge wherein a first portion of the tablet or lozenge contains the first component plus one or more additives and coformulants, and a second portion of the tablet or lozenge contains the second component plus one or more additives and coformulants.

4. A sterilising aqueous solution comprising the composition according to claim 1.

5. The solution according to claim 4, comprising the following equilibria:
NaBO₃+H²O⇄H₂O₂+sodium borate 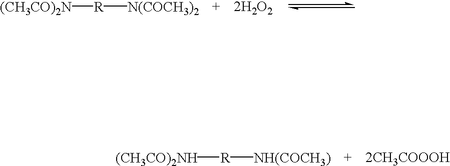
CH₃COOOH⇄CH₃COOO⁺+H⁺
where R is a C₂-C₆ alkyl group.

6. The solution according to claim 5, comprising: a pH stabilizer that may be malonic acid or phosphonic acid; an EDTA sequestrant; and a surfactant.

7. Use of a sterilising aqueous solution comprising the composition according to claim 1, for disinfecting and/or sterilising the water line of a dental unit.

8. The use according to claim 7, wherein the composition produces peracetic acid, in equilibrium with the peracetic ions, as active disinfecting and/or sterilising agent.

9. A method for disinfecting and/or sterilising a dental unit comprising the steps of:
preparing an aqueous solution comprising the composition according to claim 1;
introducing in the water line of the dental unit a portion of the aqueous solution comprising peracetic acid produced by the composition in equilibrium with the peracetic ions;
allowing the portion of aqueous solution introduced in the water line to act; and
flushing the water line with an aqueous rinsing solution.

10. The method according to claim 9, wherein the aqueous solution comprises the following equilibria:
NaBO₃+H₂O⇄H₂O₂+sodium borate 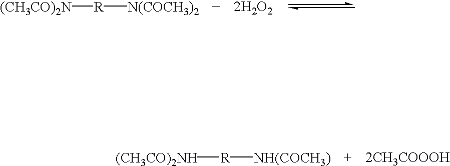
CH₃COOOH⇄CH₃COOO⁺H⁺

11. The method according to claim 10, wherein the chemical equilibrium in aqueous solution is established at a temperature of between 30 and 40° C., and at a steady pH value of between 7 and 8.

12. The method according to claim 11, wherein the aqueous solution further comprises a sequestrant such as EDTA, a pH stabilizer and a surfactant.

13. The method according to claim 12, wherein the pH stabilizer is malonic acid or phosphonic acid.

14. The method according to claim 9, wherein the portion of aqueous solution introduced in the water line is allowed to act for a period of time of between 3 and 10 minutes to accomplish the disinfection of, and between 10 and 20 minutes to accomplish the sterilization of, the inside surfaces of the water line of a dental unit.
